(19) United States
(12) Patent Application Publication
(10) Pub. No.: US 2022/0304336 A1
(43) Pub. Date: Sep. 29, 2022

(54) FEED ADDITIVE FOR REDUCING SOMATIC CELL COUNT

(71) Applicant: DSM IP ASSETS B.V., Heerlen (NL)

(72) Inventors: Cristina SIMOES CORTINHAS, Kaiseraugst (CH); Tiago SABELLA ACEDO, Kaiseraugst (CH)

(21) Appl. No.: 17/620,050

(22) PCT Filed: Jun. 11, 2020
(86) PCT No.: PCT/EP2020/066189

§ 371 Date: Dec. 16, 2021

(30) Foreign Application Priority Data
Jun. 20, 2019 (EP) 19181491.2

(51) Int. Cl. A23K 20/174; A23K 50/10

(52) U.S. Cl. CPC A23K 20/174; A23K 50/10; A23V 2250/7106

(57) ABSTRACT

Use of one or more vitamin D₃ compounds chosen from the group 1-α-hydroxy vitamin D₃, 25-hydroxy vitamin D₃, 1-α-, 25-dihydroxy vitamin D₃, to improve milk quality and/or to increase milk yield of an animal.

### FIELD OF THE INVENTION

The present invention generally relates to the field of milk producing animals. In particular, it relates to a feed supplementation composition containing one or more vitamin D₃ compounds chosen from the group 1-α-hydroxy vitamin D₃, 25-hydroxy vitamin D₃, 1-α-, 25-dihydroxy vitamin D₃. More particularly, the invention relates to a feed supplementation composition containing the above-mentioned ingredients for the improvement of milk quality and/or to increase in milk yield of a ruminant, particularly of dairy cows.

### BACKGROUND OF THE INVENTION

The production of high-quality milk is the aim of every dairy farmer. Therefore, the health of the animals, such as cows, and milk quality and yield are among their primary concerns. However, infections, such as mastitis, are rather common among cows. The somatic cell count (SCC) is commonly used as a measure of milk quality. A high SCC indicates issues of animal health and poor milk quality. Milk markets routinely rely on somatic cell counts to ensure a quality product and to assure compliance with milk quality standards. Most markets pay a bonus for low SCC and refuse high SCC milk.

High SCC lead to economic loss for the farmer due to decreased milk production, lower price for the milk, cost for treatment of the animals, discarding of poor-quality milk and holding back of milk due to antibiotic treatment. Milk yield depends on a number of factors. Generally, the lactation performance decreases towards the dry period. To improve the lactation performance, especially in the late-lactation period, further increases economic gain. It is therefore important to improve milk yield and milk quality through decreasing SCC levels.

### SUMMARY OF THE INVENTION

The present invention relates to the use of one or more vitamin D₃ compounds chosen from the group 1-α-hydroxy vitamin D₃, 25-hydroxy vitamin D₃, 1-α-, 25-dihydroxy vitamin D₃ in the manufacture of a feed composition for the improvement of milk yield and milk quality of an animal, particularly a dairy cow.

The invention relates to the use of one or more vitamin D₃ compounds chosen from the group 1-α-hydroxy vitamin D₃, 25-hydroxy vitamin D₃, 1-α-, 25-dihydroxy vitamin D₃ in the manufacture of a feed composition for the improvement of milk yield and milk quality of an animal, particularly a dairy cow.

The invention relates to the use of one or more vitamin D₃ compounds chosen from the group 1-α-hydroxy vitamin D₃, 25-hydroxy vitamin D₃, 1-α-, 25-dihydroxy vitamin D₃ in the manufacture of a feed composition for the improvement of milk yield and milk quality of an animal compared to untreated animals.

According to the present invention the improvement of milk quality is characterized by a decrease in SCC in the milk by at least 5%, more preferably by at least 10%, more preferably by at least 15% and most preferably by at least 20%, for example 20.5%.

According to the present invention the improvement of milk quality is characterized by a decrease in SCC in the milk to a value below 100,000, preferably below 90,000 cells/mL milk, for example 83′500 cells/mL.

According to the present invention the milk yield is increased by at least 0.1%, preferably 0.2%, more preferably 0.3%, more preferably 0.4%, more preferably 0.5%, more preferably 0.6%, more preferably 0.7%, for example 0.8%.

In another aspect, the invention relates to a method for the improvement of milk quality and/or to increase in milk yield of a ruminant, particularly in dairy cows, which comprises administering to an animal in need of such treatment one or more vitamin D₃ compounds chosen from the group 1-α-hydroxy vitamin D₃, 25-hydroxy vitamin D₃, 1-α-, 25-dihydroxy vitamin D₃.

In another aspect, the invention relates to a method for the decrease in SCC in milk of an animal, particularly a dairy cow, which comprises administering to an animal in need of such treatment one or more vitamin D₃ compounds chosen from the group 1-α-hydroxy vitamin D₃, 25-hydroxy vitamin D₃, 1-α-, 25-dihydroxy vitamin D₃. More specifically, the invention relates to a method for decreasing the in SCC in milk of an animal, particularly a dairy cow to a value below 100,000, preferably below 90,000 cells/mL milk, for example 83′500 cells/mL, which comprises administering to an animal in need of such treatment one or more vitamin D₃ compounds chosen from the group 1-α-hydroxy vitamin D₃, 25-hydroxy vitamin D₃, 1-α-, 25-dihydroxy vitamin D₃.

More specifically, the invention relates to a method for decreasing the in SCC in milk of an animal, particularly a dairy cow by at least 5%, more preferably by at least 10%, more preferably by at least 15% and most preferably by at least 20%, for example 20.5% compared to untreated animals, which comprises administering to an animal in need of such treatment one or more vitamin D₃ compounds chosen from the group 1-α-hydroxy vitamin D₃, 25-hydroxy vitamin D₃, 1-α-, 25-dihydroxy vitamin D₃.

More specifically, the invention relates to a method to increase in milk yield of an animal, particularly a dairy cow by at least 0.1%, preferably 0.2%, more preferably 0.3%, more preferably 0.4%, more preferably 0.5%, more preferably 0.6%, more preferably 0.7%, for example 0.8%, which comprises administering to an animal in need of such treatment one or more vitamin D₃ compounds chosen from the group 1-α-hydroxy vitamin D₃, 25-hydroxy vitamin D₃, 1-α-, 25-dihydroxy vitamin D₃.

### Definitions

Somatic cell count (SCC) is a measure of white blood cells (leucocytes) released into the udder. The number of somatic cells increases in response to an infection. The SCC is quantified as cells/mL milk. Generally, SCC values are less than 150,000 cells/mL for healthy cows.

Calcidiol: The terms “25-OH D3”, “25-hydroxy vitamin D₃” and “calcidiol” are used interchangeably.

Cholecalciferol: The terms “Cholecalciferol” and “vitamin D₃” are used interchangeably.

ROVIMIX@ Hy-D@ 1.25%: 25-hydroxy vitamin D₃ is available as a commercial formulation under the Trademark ROVIMIX@ Hy-D@ 1.25%

Ruminants: There are about 150 species of ruminants which include both domestic and wild species. Ruminants include cattle (bovines), goats, sheep and camels.

Preferred ruminants according to this invention are cattle, for example dairy cows.

Feed Additive: The term feed additive according to the invention refers to a formulation comprising one or more vitamin D₃ compounds chosen from the group 1-α-hydroxy vitamin D₃, 25-hydroxy vitamin D₃, 1-α-, 25-dihydroxy vitamin D₃ as active ingredient intended for intake by the animal.

Feed Premix: The incorporation of the composition of feed additives to animal feeds, is in practice carried out using a concentrate or a premix. A premix designates a preferably uniform mixture of one or more microingredients with diluent and/or carrier.

Premixes are used to facilitate uniform dispersion of micro-ingredients in a larger mix.

A premix according to the invention can be added to feed ingredients as solids (for example as water soluble powder) or liquids.

Feed: The term “Feed” refers to any compound, preparation, or mixture suitable for, or intended for intake by animals. The terms “feed” or “feed composition” as used herein comprise solid and liquid feed as well as drinking fluids such as drinking water. An animal feed for ruminants typically comprises high fiber (20-40% of dry matter), protein (10-20% of dry matter) and energy concentrations, such as corn silage, oat hay, cottonseeds, corn, citrus pulp, soybean meal, maize silage, grass silage, cereal grain, hay or protein meal as well as vitamins, minerals, enzymes, amino acids and/or other feed ingredients. The feed refers to a manufactured or artificial diet (i.e., formulated feed) to supplement or to replace natural feed, which is most commonly produced in form of flakes or pellets.

Such a feed typically comprises a carbohydrate source, a fiber source, a protein source, (a lipid source,) in the amounts of 15-18% (w/w) of a protein source, 35-42% (w/w) of a carbohydrate source and/or 28-33% (w/w) of a fiber source. The feed may further comprise ash, water and/or any combinations thereof. The feed may further comprise one or more ingredients selected from the group consisting of, a vitamin, mineral premix and combinations thereof.

### DETAILED DESCRIPTION OF THE INVENTION

Dairy cows are prone to infections, such as mastitis, causing a high SCC. A high SCC indicates poor milk quality and is associated with a decrease in milk yield. In accordance with the present invention it has now been found that this problem can be substantially ameliorated by administering to the animals an effective amount of one or more vitamin D₃ compounds chosen from the group 1-α-hydroxy vitamin D₃, 25-hydroxy vitamin D₃, 1-α-,25-dihydroxy vitamin D₃.

A feed supplementation composition containing one or more vitamin D₃ compounds chosen from the group 1-α-hydroxy vitamin D₃, 25-hydroxy vitamin D₃, 1-α-, 25-dihydroxy vitamin D₃ is therefore one preferred embodiment of the present invention.

In one preferred embodiment of the invention the vitamin D₃ compound is 25-hydroxy vitamin D₃, available as a commercial formulation such as under the Trademark ROVIMIX@ Hy-D@ 1.25%.

According to the present invention the preferred animal is a ruminant, preferably a cow, more preferably a dairy cow and most preferably a female Holstein cow.

According to the present invention one or more vitamin D₃ compounds are suitably administered together with the feed. Feed may be supplemented by admixing one or more of the vitamin D₃ compounds according to the present invention to regular feed or by first preparing a premix of a feed component and 25-OH D3 and subsequent mixing the premix with other feed components. The feed can be any conventional ruminant feed. The terms “feed” or “feed composition” as used herein comprise solid and liquid feed as well as drinking fluids such as drinking water.

Particularly, 25-OH D3 can be administered to the animal in form of a slow release bolus either alone or with other compounds such as rumen modifiers, ionophores, methane inhibitors etc or as a formulated powder to a premix containing other minerals, vitamins, amino acids and trace elements which premix is added to regular animal feed and thorough mixing to achieve even distribution therein.

Generally, 25-OH D3 is added to the feed in an amount required to administer from about 0.1 μg to about 5.0 μg, preferably about 0.5 μg to about 3.0 μg, more preferably about 1.0 μg to about 2.0 μg 25-OH D3 per kg body weight of an individual animal per day.

More specifically, in the manufacture of a ruminant feed in accordance with the invention, from about 1 μg/kg to about 1000 μg/kg of 25-OH D3, preferably from about 10 μg/kg to about 500 μg/kg of 25-OH D3, more preferably from about 10 μg/kg to about 100 μg/kg of 25-OH D3, most preferably from about 10 μg/kg to about 70 μg/kg of 25-OH D3 are suitably added to regular ruminant feed. Therefore, a dry feed composition for dairy cows according to the present invention contains, if it is used as the sole vitamin D₃ compound, 25-hydroxy vitamin D₃ preferably in an amount of 1 μg per kg of the dry feed composition to 1000 μg per kg of the dry feed composition, more preferably in an amount of 10 μg per kg of the dry feed composition to 750 μg per kg of the dry feed composition, more preferably in an amount of 25 μg per kg of the dry feed composition to 500 μg per kg of the dry feed composition, most preferred in an amount of 25 μg per kg of the dry feed composition to 100 μg per kg of the dry feed composition.

In a further preferred embodiment of the present invention the composition is a feed premix, i.e. one or more vitamin D₃ compounds according to the present invention are —e.g. as a formulated powder—added to other minerals, vitamins, amino acids and/or trace elements in a higher concentration in order to form the premix. For use the premix is added to and thoroughly mixed with a regular animal feed to achieve even distribution therein. Such a premix can comprise active ingredients, e.g., in a concentration of from about 1 mg/kg to about 1000 mg/kg of 25-OH D3-if 25-hydroxy vitamin D₃ is the sole vitamin D₃ compound. If one kg of such premix is added per 100 kg of regular feed this would typically meet the individual need of the animal by normal feed consumption.

For dairy cows, the dosage can range from about 5 μg to about 1000 μg per kg cattle feed diet, preferably 5 μg to about 750 μg per kg cattle feed diet, preferably from about 5 to about 80 μg 25-OH D3 per kg diet.

For dairy cows, the dosage can range from about 0.1 mg/d to about 15 mg/d, preferably 1 mg/d to about 6 mg/d.

For other ruminants, the amounts are similar, and can be based on the amount of feed given per day. For optimum results, the 25-OH supplementation should be part of a diet which contains an adequate supply of nutrients, generally known to be of benefit to the organism.

As 1-α-hydroxy vitamin D₃ and 1-α-, 25-dihydroxy vitamin D₃ are both approximately 10 times more active than 25-hydroxy vitamin D₃, their amount in a dry feed composition should be adapted accordingly. The same applies for mixtures of two or three of the vitamin D₃ compounds according to the present invention. It is most preferred to use 25-hydroxy vitamin D₃ as the sole vitamin D₃ compound.

According to the present invention it is further advantageous if the composition also contains one or more of the following ingredients: Vitamin A, Vitamin E, Biotin, copper (e.g. as CuSO₄), zinc (e.g. as ZnSO₄), cobalt (e.g. as CoSO₄), selenium (e.g. as Na₂SeO₃), iodine (e.g. as KI), manganese (e.g. as MnSO₄) and/or calcium (e.g. as CaSO₄). It is preferred to use calcium sulphate as calcium carrier in a composition according to the present invention.

When the composition is prepared in the form of a premix the premix preferably comprises from 1.5 to 6 mg 25-hydroxy vitamin D₃ (if 25-hydroxy vitamin D₃ is the sole vitamin D₃ compound), and further from 80,000 to 120,000 IU Vitamin A, from 1000 to 3000 IU Vitamin E, from 10 to 20 mg Biotin, from 200 to 300 mg copper (as CuSO₄), from 300 to 600 mg zinc (as ZnSO₄), from 5 to 10 mg cobalt (as CoSO₄), from 1 to 6 mg selenium (as Na₂SeO₃), from 5 to 10 mg iodine (as KI) and/or from 200 to 400 mg manganese (as MnSO₄) and may further comprise magnesium chloride or sulphate and sodium bicarbonate.

According to the present invention it is preferred to start administering one or more vitamin D₃ compounds chosen from the group 1-α-hydroxy vitamin D₃, 25-hydroxy vitamin D₃, 1-α-, 25-dihydroxy vitamin D₃ during lactation.

### Embodiments of the Invention can be Summarized as Follows

1. Use of one or more vitamin D₃ compounds chosen from the group 1-α-hydroxy vitamin D₃, 25-hydroxy vitamin D₃ or 1-α-,25-dihydroxy vitamin D₃ to improve milk quality and/or to increase milk yield of a ruminant.

2. The use according to claim 1, wherein the milk quality is defined by the somatic cell count in milk of a ruminant.

3. The use according to claim 2, wherein the somatic cell count is reduced by at least 5%, more preferably by at least 10%, more preferably by at least 15% and most preferably by at least 20% compared to untreated animals.

4. The use according to claim 2, wherein the somatic cell count is reduced to a value below 150,000 cells/mL milk, preferably below 100,000 cells/mL milk, most preferably below 90′000 cells/mL milk.

5. The use according to claim 1, wherein the milk yield is increased by at least 0.1%, preferably 0.2%, more preferably 0.3%, more preferably 0.4%, more preferably 0.5%, more preferably 0.6%, more preferably 0.7%, compared to untreated animals.

6. The use according to claim 1 to 5, wherein the ruminant is a dairy cow.

7. The use according to claim 6, wherein the ruminant is a female Holstein cow.

8. The use according to any of claims 1 to 7, wherein 25-hydroxy vitamin D₃ is used as sole vitamin D₃ compound.

9. The use according to any of claims 1 to 7, wherein 25-hydroxy vitamin D₃ is used in an amount from about 1 μg/kg to about 1000 μg/kg of the final feed, preferably from about 10 μg/kg to about 500 μg/kg of the final feed, more preferably from about 10 μg/kg to about 100 μg/kg of the final feed, most preferably from about 10 μg/kg to about 70 μg/kg of the final feed.

10. The use according to any of claims 1 to 7, wherein 25-hydroxy vitamin D₃ is used in an amount from about 1 mg/kg to about 1000 mg/kg of the premix.

11. Use of one or more vitamin D₃ compounds chosen from the group 1-α-hydroxy vitamin D₃, 25-hydroxy vitamin D₃, 1-α-,25-dihydroxy vitamin D₃ for the manufacture of a feed composition to improve milk quality and/or to increase milk yield of a ruminant

12. The use according to claim 11, wherein the milk quality is defined by the somatic cell count in milk of a ruminant.

13. The use according to claim 12, wherein the somatic cell count is reduced by at least 5%, more preferably by at least 10%, more preferably by at least 15% and most preferably by at least 20% compared to untreated animals.

14. The use according to claim 12, wherein the somatic cell count is reduced to a value below 150,000 cells/mL milk, preferably below 100,000 cells/mL milk, most preferably below 90′000 cells/mL milk.

15. The use according to claim 11, wherein the milk yield is increased by at least 0.1%, preferably 0.2%, more preferably 0.3%, more preferably 0.4%, more preferably 0.5%, more preferably 0.6%, more preferably 0.7%, compared to untreated animals.

16. The use according to claim 11 to 15, wherein the ruminant is a dairy cow.

17. The use according to claim 16, wherein the ruminant is a female Holstein cow.

18. The use according to any of claims 11 to 17, wherein 25-hydroxy vitamin D₃ is used as sole vitamin D₃ compound.

19. The use according to any of claims 11 to 19, wherein the feed is a dry feed composition which comprises 25-hydroxy vitamin D₃ in an amount from about 1 μg/kg to about 1000 μg/kg of the final feed, preferably from about 10 μg/kg to about 500 μg/kg of the final feed, more preferably from about 10 μg/kg to about 100 μg/kg of the final feed, most preferably from about 10 μg/kg to about 70 μg/kg of the final feed.

20. The use according to any of claims 11 to 19, wherein the feed is a premix which comprises 25-hydroxy vitamin D₃ in an amount from about 1 mg/kg to about 1000 mg/kg of the premix.

21. Method for improving milk quality and/or to increase milk yield of a ruminant compared to untreated animals said method comprising the step of administering to an animal an amount of 1 mg to 10 mg of 25-hydroxy vitamin D₃ per day.

22. The method according to claim 21, wherein the milk quality is defined by the somatic cell count in milk of a ruminant.

23. The method according to claim 22, wherein the somatic cell count is reduced by at least 5%, more preferably by at least 10%, more preferably by at least 15% and most pr method use according to claim 11, wherein the somatic cell count is reduced to a value below 150,000 cells/mL milk, preferably below 100,000 cells/mL milk, most preferably below 90′000 cells/mL milk.

24. The method according to claim 22, wherein the milk yield is increased by at least 0.1%, preferably 0.2%, more preferably 0.3%, more preferably 0.4%, more preferably 0.5%, more preferably 0.6%, more preferably 0.7%, compared to untreated animals.

25. The method according to any of claims 21 to 24, wherein the ruminant is a dairy cow.

26. The method according to claim 25 wherein the ruminant is a female Holstein cow.

27. The method according to any of claims 21 to 26, wherein 25-hydroxy vitamin D₃ is used as sole vitamin D₃ compound.

28. The method according to any of claims 21 to 27, wherein 25-hydroxy vitamin D₃ is used in an amount from about 1 μg/kg to about 1000 μg/kg of the final feed, preferably from about 10 μg/kg to about 500 μg/kg of the final feed, more preferably from about 10 μg/kg to about 100 μg/kg of the final feed, most preferably from about 10 μg/kg to about 70 μg/kg of the final feed. 29. The method according to any of claims 21 to 27, wherein 25-hydroxy vitamin D₃ is used in an amount from about 1 mg/kg to about 1000 mg/kg of the premix.

The invention is further illustrated by the following examples.

### Example 1

The study evaluated dietary 25-hydroxy vitamin D₃ (25-OH D3) at 1 mg/d per cow compared to control basal diet fed cows.

Thirty lactating Holstein cows (230 kg±137 kg liveweight at day 1; 8 primiparous) were paired blocked (15) primarily by parity and secondarily by milk yield. The cows were individually fed twice a day the same standard total mixed ration (TMR) in excess. After a 2-week adaptation to this diet the following treatments were applied over 10 weeks to each animal in a Latin Square Design:

1. no supplement (control)

2. 1 mg 25-hydroxy vitamin D₃

Treatments were applied three times per day as oral doses (capsules) before milking.

Feed was pushed up at least 10 x/d. Cows were milked 3 x/d. Daily refusal per cow was recorded. Milk yield was recorded daily. SCC was measured weekly. The DM concentrations of the corn silage and the rehydrated and ensiled corn grain were monitored weekly and TMR adjusted accordingly. The basal diet contained 1033 UI/kg of DM of cholecalciferol. The average intake of cholecalciferol was 23,800 UI/d or 35.7 UI/kg of BW. The concentration of calcidiol in plasma was increased by 25-OH D3 supplementation (117.5 vs. 52.6 ng/mL).

The results are shown in Table 1.

TABLE 1
Dry matter intake, lactation performance and
SCC on treatments Control and 25-OH D3

Treatments

| | Control | HyD |
|---|---|---|
| DMI, 84 d [kg/d] | 22.9 | 23.1 |
| DMI, d 15 to d 84 [kg/d] | 23.0 | 23.3 |
| Milk, 84 d [kg/d] | 30.9 | 31.6 |
| Milk, d 15 to d 84 [kg/d] | 30.9 | 31.7 |
| SCC [cells/mL] | 105,000 | 83,500 |

The 25-OH D3 treatment resulted in an increased milk yield (31.7 vs. 30.9 kg/d) and reduced milk SCC from 105,000 cells/mL to 83,500 cells/mL at similar DMI (23.0 kg/d).

### Example 2

A feed formulation for dairy cows can be prepared as follows (Dry Matter basis):

| Ingredients | % by weight |
|---|---|
| Corn silage | 45.0 |
| Oat hay | 4.0 |
| Whole cottonseeds | 8.3 |
| Finely ground mature corn | 14.0 |
| Citrus pulp | 8.1 |
| Soybean meal | 13.5 |
| Low ruminal degradability soybean meal (Soypass) | 4.0 |
| Vitamins and trace elements premix¹ | 3.1 |

¹composition:
11.86% of Ca (analyzed)
3.93% of P (analyzed)
3.80% Mg
5.0% K
1.5% S
10.6% Na
7.4% Cl
11.5 mg/kg Co
417 mg/kg Cu (as CuSO₄)
1165 mg/kg Mn (as MnSO₄)
2330 mg/kg Zn (as ZnSO₄)
15 mg/kg Se (as Na₂SeO₃)
17.5 mg/kg I (as KI)
116,666 UI/kg Vitamin A
33,333 UI/kg Vitamin D
833 UI/kg Vitamin E

The ingredients are mixed together.

1. Use of one or more vitamin D₃ compounds chosen from the group 1-α-hydroxy vitamin D₃, 25-hydroxy vitamin D₃ or 1-α-,25-dihydroxy vitamin D₃ to improve milk quality and/or to increase milk yield of a ruminant.

2. The use according to claim 1, wherein the milk quality is defined by the somatic cell count in milk of a ruminant.

3. The use according to claim 2, wherein the somatic cell count is reduced by at least 5%, more preferably by at least 10%, more preferably by at least 15% and most preferably by at least 20% compared to untreated animals.

4. The use according to claim 2, wherein the somatic cell count is reduced to a value below 150,000 cells/mL milk, preferably below 100,000 cells/mL milk, most preferably below 90′000 cells/mL milk.

5. The use according to claim 1, wherein the milk yield is increased by at least 0.1%, preferably 0.2%, more preferably 0.3%, more preferably 0.4%, more preferably 0.5%, more preferably 0.6%, more preferably 0.7%, compared to untreated animals.

6. The use according to claim 1, wherein 25-hydroxy vitamin D₃ is used in an amount from about 1 μg/kg to about 1000 μg/kg of the final feed, preferably from about 10 μg/kg to about 500 μg/kg of the final feed, more preferably from about 10 μg/kg to about 100 μg/kg of the final feed, most preferably from about 10 μg/kg to about 70 μg/kg of the final feed.

7. Use of one or more vitamin D₃ compounds chosen from the group 1-α-hydroxy vitamin D₃, 25-hydroxy vitamin D₃, 1-α-,25-dihydroxy vitamin D₃ for the manufacture of a feed composition to improve milk quality and/or to increase milk yield of a ruminant.

8. The use according to claim 7, wherein the milk quality is defined by the somatic cell count in milk of a ruminant.

9. The use according to claim 8, wherein the somatic cell count is reduced by at least 5%, more preferably by at least 10%, more preferably by at least 15% and most preferably by at least 20% compared to untreated animals.

10. The use according to claim 8, wherein the somatic cell count is reduced to a value below 150,000 cells/mL milk, preferably below 100,000 cells/mL milk, most preferably below 90′000 cells/mL milk.

11. The use according to claim 7, wherein the feed is a dry feed composition which comprises 25-hydroxy vitamin D₃ in an amount from about 1 g/kg to about 1000 μg/kg of the final feed, preferably from about 10 μg/kg to about 500 g/kg of the final feed, more preferably from about 10 μg/kg to about 100 μg/kg of the final feed, most preferably from about 10 μg/kg to about 70 μg/kg of the final feed.

12. Method for improving milk quality and/or to increase milk yield of a ruminant compared to untreated animals said method comprising the step of administering to an animal an amount of 1 mg to 10 mg of 25-hydroxy vitamin D₃ per day.

13. The method according to claim 12, wherein the milk quality is defined by the somatic cell count in milk of a ruminant.

14. The method according to claim 13, wherein the somatic cell count is reduced by at least 5%, more preferably by at least 10%, more preferably by at least 15% and, wherein the somatic cell count is reduced to a value below 150,000 cells/mL milk, preferably below 100,000 cells/mL milk, most preferably below 90′000 cells/mL milk.

15. The method according to claim 12, wherein 25-hydroxy vitamin D₃ is used in an amount from about 1 μg/kg to about 1000 μg/kg of the final feed, preferably from about 10 μg/kg to about 500 μg/kg of the final feed, more preferably from about 10 μg/kg to about 100 μg/kg of the final feed, most preferably from about 10 μg/kg to about 70 μg/kg of the final feed.
